(19) United States
(12) Patent Application Publication
(10) Pub. No.: US 2004/0266002 A1
(43) Pub. Date: Dec. 30, 2004

(54) Use of abscisic acid in somatic embryogenesis of pine trees

(75) Inventor: Carolyn Carpenter, Seattle, WA (US)

Correspondence Address: WEYERHAEUSER COMPANY, INTELLECTUAL PROPERTY DEPT., CH 1J27, P.O. BOX 9777, FEDERAL WAY, WA, 98063, US

(73) Assignee: Weyerhaeuser Company

(21) Appl. No.: 10/859,881

(22) Filed: Jun. 2, 2004

Related U.S. Application Data
(60) Provisional application No. 60/484,163, filed on Jun. 30, 2003.

(51) Int. Cl. C12N 5/04

(52) U.S. Cl. 435/422

(57) ABSTRACT

One aspect of the invention provides methods for producing cotyledonary pine embryos. The methods of the invention comprise the steps of: (a) culturing pre-cotyledonary pine somatic embryos in a development medium comprising abscisic acid and an adsorbent composition; and (b) culturing the pre-cotyledonary pine somatic embryos treated in accordance with step (a) in a development medium that does not include abscisic acid to yield cotyledonary pine somatic embryos.

### FIELD OF THE INVENTION

The present invention relates to methods for producing plant embryos in vitro, and optionally producing plants from the plant embryos.

### BACKGROUND OF THE INVENTION

The demand for coniferous trees, such as pines and firs, to make wood products continues to increase. One proposed solution to this problem is to identify individual trees that possess desirable characteristics, such as a rapid rate of growth, and produce numerous, genetically identical, clones of the superior trees by somatic cloning.

Somatic cloning is the process of creating genetically identical trees from tree tissue other than the male and female gametes. In one approach to somatic cloning, plant tissue is cultured in an initiation medium which includes hormones, such as auxins and/or cytokinins, that initiate formation of embryogenic cells that are capable of developing into somatic embryos. The embryogenic cells are then further cultured in a maintenance medium that promotes multiplication of the embryogenic cells to form pre-cotyledonary embryos (i.e., embryos that do not possess cotyledons). The multiplied embryogenic cells are then cultured in a development medium that promotes development of cotyledonary somatic embryos which can, for example, be placed within artificial seeds and sown in the soil where they germinate to yield conifer seedlings. The seedlings can be transplanted to a growth site for subsequent growth and eventual harvesting to yield lumber, or wood-derived products.

There is a continuing need to improve the efficiency of somatic cloning of conifer embryos in order to increase production of cotyledonary somatic embryos that are capable of germinating to yield pine plants. Preferably the conifer somatic embryos, formed in vitro, are physically and physiologically similar, or identical, to conifer zygotic embryos formed in vivo in conifer seeds. The present invention provides methods that address this need with respect to conifers of the genus Pinus.

### SUMMARY OF THE INVENTION

One aspect of the invention provides methods for producing cotyledonary pine somatic embryos. The methods of the invention comprise the steps of: (a) culturing pre-cotyledonary pine somatic embryos in a development medium comprising abscisic acid and an adsorbent composition; and (b) culturing the pre-cotyledonary pine somatic embryos treated in accordance with step (a) in a development medium that does not include abscisic acid to yield cotyledonary pine somatic embryos.

In some embodiments, pre-cotyledonary pine somatic embryos are cultivated in a development medium containing abscisic acid and an adsorbent composition for between about four weeks and about six weeks, after which the embryos are transferred to a development medium without abscisic acid to form cotyledonary somatic embryos. The abscisic acid concentration in the development medium containing abscisic acid may be from about 1 mg/L to about 100 mg/L, such as from about 10 mg/L to about 50 mg/L. The concentration of adsorbent composition in the development medium containing abscisic acid may be from about 0.1 g/L and 5 g/L.

The present invention also provides cotyledonary pine somatic embryos. The cotyledonary pine somatic embryos produced according to the methods of the invention are typically larger and have a greater dry weight (e.g., weigh at least 20% more, such as between 20% and 30% more) than cotyledonary pine somatic embryos produced by a reference method that does not include the steps of (a) culturing pre-cotyledonary pine somatic embryos in a development medium comprising abscisic acid and an adsorbent composition; and (b) culturing the pre-cotyledonary pine somatic embryos treated in accordance with step (a) in a development medium that does not include abscisic acid to yield cotyledonary pine somatic embryos.

The methods of the present invention are useful for preparing cotyledonary pine somatic embryos that can be further characterized, such as by genetic or biochemical means, and/or can be germinated to yield pine trees, if so desired. Thus, for example, the methods of the invention can be used to produce clones of individual pine trees that possess one or more desirable characteristics, such as a rapid growth rate or improved wood quality. For example, a population of mature pine somatic embryos prepared in accordance with the present invention can be used to produce a stand, or forest, of pine trees possessing one or more desirable characteristics, such as a rapid growth rate or improved wood quality. The trees can be utilized to produce wood products.

### DETAILED DESCRIPTION OF THE PREFERRED EMBODIMENT

Unless specifically defined herein, all terms used herein have the same meaning as they would to one skilled in the art of the present invention.

Unless stated otherwise, all concentration values that are expressed as percentages are weight per volume percentages.

In one aspect, the present invention provides methods for producing cotyledonary pine somatic embryos. The methods of the invention comprise the steps of: (a) culturing pre-cotyledonary pine somatic embryos in a development medium comprising abscisic acid and an adsorbent composition; and (b) culturing the pre-cotyledonary pine somatic embryos treated in accordance with step (a) in a development medium that does not include abscisic acid to yield cotyledonary pine somatic embryos. It will be understood that, in the practice of the present invention, reference to culturing somatic embryos, or other plant tissue, in a medium (e.g., culturing pre-cotyledonary pine somatic embryos in a development medium) encompasses all of the following culturing methods: (1) culturing the embryos, or other plant tissue, on the surface of a solid, or semisolid medium; (2) partly immersing the embryos, or other plant tissue, in a liquid, solid or semisolid medium (e.g., inserting part of an embryo into a solid or semisolid medium so that part of the embryo is disposed within the medium); and (3) completely immersing the embryos, or other plant tissue, in a liquid medium.

As used herein, the term “cotyledonary pine somatic embryo” refers to a pine somatic embryo having a well defined elongated bipolar structure with latent meristem with cotyledonary primordia at one end and a potential radicle at the opposite end. The cotyledonary structure frequently appears as a small “crown” at one end of the embryo. A cotyledonary pine somatic embryo is capable of germinating to form a pine plant.

As used herein, the term “pre-cotyledonary pine somatic embryo” refers to a pine somatic embryo in which the cotyledonary primordia have not yet formed. A pre-cotyledonary pine somatic embryo is not capable of germinating to form a pine plant.

The methods of the invention can be used to produce cotyledonary somatic embryos from any member of the genus Pinus, such as Loblolly pine (Pinus taeda).

Pre-cotyledonary pine somatic embryos can be prepared from pine somatic cells, such as cells obtained from pine zygotic embryos. For example, cells from pine zygotic embryos can be induced by hormones to form embryonal suspensor cell masses (ESMs) that can be treated in accordance with the present invention to yield cotyledonary somatic embryos. ESMs can be prepared, for example, from pre-cotyledonary embryos removed from seed. For example, the seed are surface sterilized before removing the pre-cotyledonary embryos which are then cultured in an induction medium that promotes formation of ESMs which include early stage embryos in the process of multiplication by budding and cleavage. Suitable induction media are exemplified by BM₁ set forth in EXAMPLE 1 herein and are further described below.

According to the methods of the invention, pre-cotyledonary pine somatic embryos are cultured in a development medium comprising abscisic acid and an adsorbent composition. Abscisic acid is a sesquiterpenoid plant hormone that is implicated in a variety of plant physiological processes (see, e.g., Milborrow (2001) J. Exp. Botany 52:1145-1164; Leung & Giraudat (1998) Ann. Rev. Plant Physiol. Plant Mol. Biol. 49:199-123). In some embodiments of the methods of the invention, the concentration of abscisic acid in the development medium is between 0.5 mg/L and 500 mg/L. In some embodiments of the methods of the invention, the concentration of abscisic acid in the development medium is between 1 mg/L and 100 mg/L. In some embodiments of the methods of the invention, the concentration of abscisic acid in the development medium is between 5 mg/L and 50 mg/L. In some embodiments of the methods of the invention, the concentration of abscisic acid in the development medium is between 5 mg/L and 25 mg/L.

The development medium is typically a solid medium. The development medium typically contains nutrients that sustain the pre-cotyledonary embryos. Maltose may be included in the development medium as the principal or sole source of sugar for the embryos. Useful maltose concentrations are within the range of from about 1% to about 2.5%.

Suitable development media typically do not include growth-promoting hormones, such as auxins and cytokinins. The development medium may contain gellan gum, typically ranging in concentration from about 0% to about 0.35%. The osmolality of the development medium can be adjusted to a value that falls within a desired range, such as from about 250 mM/Kg to about 450 mM/Kg. Typically, an osmolality of 350 mM or higher is advantageous. An example of a suitable development medium is medium BM₃ set forth in EXAMPLE 1 herein.

In the practice of the present invention, pre-cotyledonary pine somatic embryos may be cultured in a development medium containing abscisic acid and an adsorbent composition for a period of from 4 weeks to 6 weeks, at a temperature of from 10° C. to 30° C., such as from 15° C. to 25° C., or such as from 20° C. to 23° C.

The adsorbent composition can be any composition (or combination of adsorbent compositions) that is not toxic to the embryogenic cells at the concentrations utilized in the practice of the present methods, and that is capable of adsorbing abscisic acid, and toxic compounds produced by the plant cells during embryo development, that are present in the medium. Non-limiting examples of useful adsorbent compositions include activated charcoal, soluble poly(vinyl pyrrolidone), insoluble poly(vinyl pyrrolidone), activated alumina, and silica gel. The adsorbent composition may be present in an amount, for example, of from about 0.1 g/L to about 5 g/L. When a combination of adsorbent compositions is used the total concentration of adsorbent compositions is typically from about 0.1 g/L to about 5 g/L.

After cultivating the pre-cotyledonary pine somatic embryos on a development medium containing abscisic acid and an adsorbent composition, the abscisic-acid treated pre-cotyledonary embryos are transferred to a development medium without abscisic acid, in which the pre-cotyledonary embryos continue their development into cotyledonary embryos. Typically, the composition of the development medium without abscisic acid is similar, or identical, to that of the development medium with abscisic acid, except that it does not include abscisic acid.

Abscisic acid-treated pre-cotyledonary pine somatic embryos may be cultured in a development medium without abscisic acid for a period of, for example, about 6 weeks to 8 weeks, at a temperature of from 10° C. to 30° C., such as from 15° C. to 25° C., or such as from 20° C. to 23° C.

In some embodiments, the present invention provides methods for producing cotyledonary pine somatic embryos, the methods each comprising the steps of (a) culturing pine somatic cells in an induction medium to yield embryogenic cells; (b) culturing the embryogenic cells prepared in step (a) in a maintenance medium to multiply the embryogenic cells and form pre-cotyledonary pine somatic embryos; (c) culturing pre-cotyledonary pine somatic embryos formed in step (b) in a development medium comprising abscisic acid and an adsorbent composition; and (d) culturing the pre-cotyledonary pine somatic embryos treated in accordance with step (c) in a development medium that does not include abscisic acid to yield cotyledonary pine somatic embryos.

The induction medium typically includes inorganic salts and organic nutrient materials. The osmolality of the induction medium is typically about 160 mM/kg or even lower, but it may be as high as 170 mM/kg. The induction medium typically includes growth hormones. Examples of hormones that can be included in the induction medium are auxins (e.g., 2,4-dichlorophenoxyacetic acid (2,4-D)) and cytokinins (e.g., 6-benzylaminopurine (BAP)). Auxins can be utilized, for example, at a concentration of from 1 mg/L to 200 mg/L. Cytokinins can be utilized, for example, at a concentration of from 0.1 mg/L to 10 mg/L.

The induction medium may contain an adsorbent composition, especially when very high levels of growth hormones are used. The adsorbent composition can be any composition that is not toxic to the embryogenic cells at the concentrations utilized in the practice of the present methods, and that is capable of absorbing growth-promoting hormones, and toxic compounds produced by the plant cells during embryo development, that are present in the medium. Non-limiting examples of useful adsorbent compositions include activated charcoal, soluble poly(vinyl pyrrolidone), insoluble poly(vinyl pyrrolidone), activated alumina, and silica gel. The adsorbent composition may be present in an amount, for example, of from about 0.1 g/L to about 5 g/L.

An example of an induction medium useful in the practice of the present invention is medium BM₁, set forth in EXAMPLE 1 herein.

Pine somatic cells are typically cultured in an induction medium for a period of from 4 weeks to 14 weeks, such as from 6 weeks to 10 weeks, or such as from 7 weeks to 8 weeks, at a temperature of from 10° C. to 30° C., such as from 15° C. to 25° C., or such as from 20° C. to 23° C.

The maintenance medium may be a solid medium, or it may be a liquid medium which can be agitated to promote growth and multiplication of the embryogenic tissue. The osmolality of the maintenance medium is typically higher than the osmolality of the induction medium, typically in the range of 180-400 mg. The maintenance medium may contain nutrients that sustain the embryogenic tissue, and may include hormones, such as one or more auxins and/or cytokinins, that promote cell division and growth of the embryogenic tissue. Typically, the concentrations of hormones in the maintenance medium are lower than their concentration in the induction medium.

It is generally desirable, though not essential, to include maltose as the sole, or principal, metabolizable sugar source in the maintenance medium. Examples of useful maltose concentrations are within the range of from about 1% to about 2.5%. An example of a suitable maintenance medium is medium BM₂ set forth in EXAMPLE 1 herein.

Pine embryogenic cells are typically cultured in, or on, a maintenance medium for a period of from 4 weeks to 16 weeks, such as from 6 weeks to 10 weeks, or such as from 7 weeks to 8 weeks, at a temperature of from 10° C. to 30° C., such as from 15° C. to 25° C., or such as from 20° C. to 23° C.

Pre-cotyledonary pine somatic cells formed from pine embryogenic cells are treated with abscisic acid, in the presence of at least one adsorbent composition, to produce treated pre-cotyledonary pine somatic embryos, as described above. Treated pre-cotyledonary embryos are then cultured in the absence of abscisic acid to form cotyledonary pine somatic embryos.

In some embodiments, cotyledonary somatic embryos produced according to the methods of the invention are cultivated in at least one maturation and stratification medium to further mature the cotyledonary pine somatic embryos and improve their germination frequency. The embryos are typically incubated in stratification medium at a temperature in the range of from 1° C. to 10° C.

The maturation medium can be a liquid or a solid medium. The stratification medium is typically a liquid medium. The maturation medium and stratification medium also may include nutrients that sustain the incubated cotyledonary embryos and/or maturing embryos, and one or more agents for adjusting the osmolality of the medium to within a desired range. An exemplary osmolality range for maturation medium is from 250 to 450 mM/Kg. An exemplary osmolality range for stratification medium is from 131 to 148.mM/Kg. The pH of the maturation medium and stratification medium can also be adjusted to a desired value. An exemplary pH range for maturation medium is from 5.6 to 5.8. An exemplary pH range for stratification medium is from 5.3 to 6.4. Maltose may be included in the maturation medium or stratification medium as the principal or sole source of metabolizable sugar. Useful maltose concentrations are within the range of about 1% to about 2.5%.

The maturation or stratification medium may contain an adsorbent composition, such as activated charcoal, as described above for the induction medium. An exemplary stratification medium is BM₄ set forth in EXAMPLE 1.

Cotyledonary pine somatic embryos are typically cultured in a maturation medium for a period of from 4 weeks to 16 weeks, such as from 6 weeks to 10 weeks, or such as from 8 weeks to 12 weeks, at a temperature of from 10° C. to 30° C., such as from 15° C. to 25° C., or such as from 20° C. to 23° C.

The methods of the invention typically produce cotyledonary pine somatic embryos having a higher survival rate during germination, and that are larger than cotyledonary pine somatic embryos produced by a method wherein abscisic acid is present throughout the developmental stage. Thus, the cotyledonary pine somatic embryos produced according to the methods of the invention typically have a greater dry weight (e.g., weigh 20% to 30% more) than cotyledonary pine somatic embryos produced by a reference method that does not include the steps of (a) culturing pre-cotyledonary pine somatic embryos in a development medium comprising abscisic acid and an adsorbent composition; and (b) culturing the pre-cotyledonary pine somatic embryos treated in accordance with step (a) in a development medium that does not include abscisic acid to yield cotyledonary pine somatic embryos.

The methods of the invention can be used, for example, to produce clones of individual pine trees that possess one or more desirable characteristics, such as a rapid growth rate. Thus, in one aspect, the present invention provides methods for producing a population of genetically-identical, mature pine somatic embryos. The methods of this aspect of the invention each include the steps of: (a) culturing pre-cotyledonary pine somatic embryos in a development medium comprising abscisic acid and an adsorbent composition; and (b) culturing the pre-cotyledonary pine somatic embryos treated in accordance with step (a) in a development medium that does not include abscisic acid to yield cotyledonary pine somatic embryos, wherein substantially all of the embryos are genetically identical. It will be understood by those of skill in the art that the term “genetically identical” encompasses pine somatic embryos that are derived from cells having identical genetic material, but which have acquired some minor genetic differences (e.g., single base changes in genomic DNA) as a result of the somatic cloning process.

Another aspect of the invention provides cotyledonary pine somatic embryos generated using the methods of the invention. The cotyledonary pine somatic embryos produced using the methods of the invention can optionally be germinated to form pine plants which. can be grown into pine trees, if desired. Alternatively, the embryos may be disposed within artificial seeds for subsequent germination. The cotyledonary pine somatic embryos can be germinated, for example, on a solid germination medium, such as medium BM₅ medium set forth in EXAMPLE 1 herein. The germinated plants can be transferred to soil for further growth. For example, the germinated plants can be planted in soil in a greenhouse and allowed to grow before being transplanted to an outdoor site. Typically, the cotyledonary pine somatic embryos are illuminated to stimulate germination. Typically, all the steps of the methods of the invention, except germination, are conducted in the dark.

The following examples merely illustrate the best mode now contemplated for practicing the invention, but should not be construed to limit the invention.

### EXAMPLE 1

This Example shows a representative method for producing cotyledonary Loblolly pine somatic embryos.

Female gametophytes containing zygotic embryos were removed from seeds four to five weeks after fertilization. The seed coats were removed but the embryos were not further dissected out of the surrounding gametophyte other than to excise the nucellar end. The cones were stored at 4° C. until used. Immediately before removal of the immature embryos the seeds were sterilized utilizing an initial washing and detergent treatment followed by a ten minute sterilization in 15% H₂O₂. The explants were thoroughly washed with sterile distilled water after each treatment.

Tables 1 and 2 set forth the compositions of media useful for producing Loblolly pine somatic embryos.

TABLE 1
Pinus Taeda Basal Medium (BM)

| | Concentration | |
|---|---|---|
| Constituent | (mg/L) | |
| NH₄NO₃ | 150.0 | |
| KNO₃ | 909.9 | |
| KH₂PO₄ | 136.1 | |
| Ca(NO₃)₂.4H₂O | 236.2 | |
| CaCl₂.4H₂O | 50.0 | |
| MgSO₄.7H₂O | 246.5 | |
| Mg(NO₃)₂.6H₂O | 256.5 | |
| MgCl₂.6H₂O | 50.0 | |
| KI | 4.15 | |
| H₃BO₃ | 15.5 | |
| MnSO₄.H₂O | 10.5 | |
| ZnSO₄.7H₂O | 14.4 | |
| NaMoO₄.2H₂O | 0.125 | |
| CuSO₄.5H₂O | 0.125 | |
| CoCl₂.6H₂O | 0.125 | |
| FeSO₄.7H₂O | 13.9 | |
| Na₂EDTA | 18.65 | |
| Maltose | 20,000 to | |
| | 30,000. | |
| myo-Inositol | 200 | |
| Casamino acids | 500 | |
| L-Glutamine | 1000 | |
| Thiamine.HCl | 1.00 | |
| Pyridoxine.HCl | 0.50 | |
| Nicotinic acid | 0.50 | |
| Glycine | 2.00 | |
| Gelrite | 2.5 | g/L |
| pH adjusted to 5.7 | | |

TABLE 2
Composition of Media for Different Stage Treatments

| | |
|---|---|
| BM₁-Induction | BM + 2,4-D (15 μM) + Kinetin (2 μM) + BAP (2 μM) |
| Medium | |
| BM₂- | BM + 2,4-D(1 mg/L) + Kinetin (0.1 mg/L) + BAP |
| Maintenance | (0.1 mg/L), FeSO₄.7H₂O increased to 27.56 mg/L, |
| Medium | Na₂EDTA increased to 37.3 mg/L. |
| | Maltose is substituted for sucrose on an equal weight |
| | basis. Gelrite is added when a solid medium is desired. |
| BM₃- | BM + 13% PEG-8000 + 2% maltose, myo-inositol |
| Development | concentration increased to 1000 mg/L, + 0.1% activated |
| Medium | charcoal + gellan gum (0.25%). Maltose is substituted |
| | for sucrose at 2%. The following amino acid mixture is |
| | added: L-proline (100 mg/L), L-asparagine (100 mg/L), |
| | L-arginine (50 mg/L), L-alanine (20 mg/L), and |
| | L-serine (20 mg/L). Gelrite is added when a solid |
| | medium is desired. |
| BM₄- | BM₃ modified by omitting abscisic acid and PEG. |
| Stratification | |
| Medium | |
| BM₅- | BM modified by replacing maltose with sucrose at |
| Germination | 20,000 mg/L, myo-inositol concentration decreased to |
| Medium | 100 mg/L, glutamine and casamino acids are omitted, |
| | Gelrite replaced with 8 g/L agar, and 0.25% activated |
| | charcoal. |

Induction: Sterile gametophytes with intact embryos were placed on a solid BM₁ culture medium and held in an environment at 22°-25 C with a 24 hour dark photoperiod for a time of 3-5 weeks. The length of time depends on the particular genotype being cultured. At the end of this time a white mucilaginous mass formed in association with the original explants. Microscopic examination typically reveals numerous early stage embryos associated with the mass. These are generally characterized as having a long thin-walled suspensor associated with a small head with dense cytoplasm and large nuclei.

Osmolality of the induction medium may in some instances be as high as 170 mM/kg. Normally it is about 160 mM/kg or even lower (such as 150 mM/kg).

Maintenance and Multiplication: Early stage embryos removed from the masses generated in the induction stage were first placed on a BM₂ gelled maintenance and multiplication medium. This differs from the induction medium in that the growth hormones (both auxins and cytokinins) are reduced by at least a full order of magnitude. Osmolality of this medium is typically raised from that of the induction medium to about 180 mM/kg or higher (typically within the range of about 180-400 mM/kg for Pinus taeda) by increasing the concentration of myo-inositol to 0.02% w/v. The temperature and photoperiod were again 22°-25° C. with 24 hours in the dark. Embryos were cultured 12-14 days on the BM₂ solid medium before transferring to a liquid medium for further subculturing. This liquid medium has the same composition as BM₂, but lacks the gellant. The embryos at the end of the solid maintenance stage were typically similar in appearance to those from the induction stage. After 5 to 6 weekly subcultures on the liquid maintenance medium pre-cotyledonary embryos have formed. These are characterized by smooth embryonal heads, estimated to typically have over 100 individual cells, with multiple suspensors.

Development: Pre-cotyledonary somatic embryos were transferred from maintenance medium into solid development medium BM₃ containing 25 mg/L abscisic acid for four weeks to yield treated pre-cotyledonary somatic embryos. The development medium either lacks growth hormones entirely, or has them present only at very low levels. The further inclusion of an adsorbent composition in this medium is advantageous. The adsorbent composition is normally present at a concentration of about 0.1-5 g/L, more generally about 0.25-2.5 g/L. Gelrite was included at a concentration of about 0.25%.

The osmotic potential of the development medium may be raised substantially over that of the maintenance medium. It has been found advantageous to have an osmolality as high as 350 mM/kg or even higher.

After culturing pre-cotyledonary embryos on solid development medium containing 25 mg/L abscisic acid for 4 weeks, the abscisic acid-treated pre-cotyledonary embryos were transferred on a filter paper support to solid development medium without abscisic acid. The solid development medium without abscisic acid had the same composition as development medium BM₃. Development is preferably carried out in complete darkness at a temperature of 22°-25 C until elongated cotyledonary embryos have developed. Development time is typically several weeks, such as 10 to 12 weeks.

Maturation and Stratification: Cotyledonary embryos are singulated and transferred to stratification medium BM₄ for 4 weeks. This medium is similar to development medium but lacks abscisic acid, PEG and gellan gum.

Drying: The mature embryos still on their filter paper support were lifted from the pad and placed in a closed container over water, at a relative humidity of 97% to 99%, for a period of about three weeks.

Germination: The dried mature embryos were rehydrated by placing them, while still on the filter paper support, for about 24 hours on a pad saturated with liquid germination medium. The embryos were then placed individually on solid BM₅ medium for germination. This is a basal medium lacking growth hormones which has been modified by reducing myo-inositol and organic nitrogen, and replacing maltose with sucrose. The embryos were incubated on BM₅ medium for about 12 weeks under environmental conditions of 23°-25° C., and a 16 hour light—8 hour dark photoperiod, until the resulting plantlets have a well developed radicle and hypocotyl and green cotyledonary structure and epicotyl.

Because of the reduced carbohydrate concentration, the osmotic potential of the germination medium is further reduced below that of the development medium. It is normally below about 150 mM/kg (such as about 125 mM/kg).

### EXAMPLE 2

This Example shows the effects on the production of Loblolly pine (Pinus taeda) cotyledonary somatic embryos of treating pre-cotyledonary somatic embryos with a pulse of abscisic acid.

Female gametophytes containing zygotic embryos were removed from seeds of genotypes A, B and C, as described in EXAMPLE 1, above. The induction and maintenance stages were as described in EXAMPLE 1.

To test the effect of a pulse of abscisic acid, pre-cotyledonary embryos were transferred to a filter paper support placed on solid development medium BM₃ containing abscisic acid. Six different treatments were tested. The concentration of abscisic acid, and the time period during which the pre-cotyledonary embryos were exposed to the abscisic acid during each treatment, are shown in Table 3.

TABLE 3
Concentration (mg/L) of Abscisic Acid Used in Treatments 1 Thru 6

| | Number of weeks after plating | | | | | | | | | | | | | | |
|---|---|---|---|---|---|---|---|---|---|---|---|---|---|---|---|
| Treatment | 1 | 2 | 3 | 4 | 5 | 6 | 7 | 8 | 9 | 10 | 11 | 12 | 12-16 | 16-19 | 19+ |
| Control | 25 | 25 | 25 | 25 | 25 | 25 | 25 | 25 | 25 | 25 | 25 | 25 | cold | dry | germ |
| 1 | 25 | 25 | 25 | 0 | 0 | 0 | 0 | 0 | 0 | 0 | 0 | 0 | cold | dry | germ |
| 2 | 25 | 25 | 25 | 25 | 25 | 0 | 0 | 0 | 0 | 0 | 0 | 0 | cold | dry | germ |
| 3 | 25 | 25 | 25 | 25 | 25 | 25 | 25 | 0 | 0 | 0 | 0 | 0 | cold | dry | germ |
| 4 | 25 | 25 | 25 | 25 | 25 | 25 | 25 | 25 | 25 | 0 | 0 | 0 | cold | dry | germ |
| 5 | 25 | 25 | 25 | 15 | 15 | 10 | 10 | 5 | 5 | 0 | 0 | 0 | cold | dry | germ |
| 6 | 25 | 25 | 25 | 25 | 25 | 15 | 15 | 10 | 10 | 0 | 0 | 0 | cold | dry | germ |

The term “cold” in Table 3 refers to stratification treatment.
The term “dry” in Table 3 refers to drying the embryos at a relative humidity of 97% to 99%.

As a control, pre-cotyledonary embryos were maintained in development medium BM₃ containing 25 mg/L abscisic acid for twelve weeks. After twelve weeks, both control and experimental embryos were taken through the standard post-development treatments as described in EXAMPLE 1. Germination was assessed after twelve weeks on germination media in the light at room temperature.

At the end of development, both control and experimental embryos showed good morphological form, good cotyledon development, and normal color. The embryos receiving the abscisic acid pulse had a higher dry weight than the control embryos, as shown in Table 4 (asterisks indicate values that are statistically significantly higher than the control value).

TABLE 4
Dry Weight of Loblolly Pine Embryos Treated
With Abscisic Acid Pulse

| | | Mean Dry Weight |
|---|---|---|
| Genotype | Treatment | (mg/emb) |
| A | Control | 0.27 |
| | 1 | 0.38* |
| | 2 | 0.36* |
| | 3 | 0.34 |
| | 4 | 0.32 |
| | 5 | 0.36* |
| | 6 | 0.33 |
| B | Control | 0.31 |
| | 1 | 0.32 |
| | 2 | 0.31 |
| | 3 | 0.37 |
| | 4 | 0.37 |
| | 5 | 0.36 |
| | 6 | 0.36 |
| C | Control | 0.55 |
| | 1 | 0.79* |
| | 2 | 0.80* |
| | 3 | 0.69 |
| | 4 | 0.76* |
| | 5 | 0.90* |
| | 6 | 0.84* |
| All genotypes | Control | 0.37 |
| combined | 1 | 0.49* |
| | 2 | 0.47* |
| | 3 | 0.47* |
| | 4 | 0.48* |
| | 5 | 0.50* |
| | 6 | 0.48* |

Asterisks (*) represent values which are statistically different than the control treatment (95% confidence level).

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for producing cotyledonary pine somatic embryos, comprising the steps of:
(a) culturing pre-cotyledonary pine somatic embryos in a development medium comprising abscisic acid and an adsorbent composition; and
(b) culturing the pre-cotyledonary pine somatic embryos treated in accordance with step (a) in a development medium that does not include abscisic acid to yield cotyledonary pine somatic embryos.

2. The method of claim 1, wherein the pre-cotyledonary pine somatic embryos are cultured in a development medium comprising abscisic acid and an adsorbent composition for between about four weeks and about six weeks.

3. The method of claim 1, wherein the pre-cotyledonary pine somatic embryos are cultured in a development medium comprising abscisic acid and an adsorbent composition for about four weeks.

4. The method of claim 1, wherein the concentration of the abscisic acid in the development medium of step (a) is between about 1 mg/L and 100 mg/L.

5. The method of claim 1, wherein the concentration of the abscisic acid in the development medium of step (a) is between about 10 mg/L and 50 mg/L.

6. The method of claim 1, wherein the cotyledonary pine somatic embryos have a dry weight that is at least 20% greater than the dry weight of cotyledonary pine somatic embryos produced by a reference method that does not include steps (a) and (b).

7. The method of claim 1, wherein the cotyledonary pine somatic embryos have a dry weight that is between 20% and 30% greater than the dry weight of cotyledonary pine somatic embryos produced by a reference method that does not include steps (a) and (b).

8. The method of claim 1, wherein the concentration of the adsorbent composition in the development medium of step (a) is between about 0.1 g/L and 5 g/L.

9. The method of claim 1 wherein the adsorbent composition is selected from the group consisting of activated charcoal, soluble poly(vinyl pyrrolidone), insoluble poly(vinyl pyrrolidone), activated alumina, and silica gel.

10. The method of claim 1 wherein the adsorbent composition is activated charcoal.

11. A method for producing cotyledonary pine somatic embryos, the method comprising the steps of:
(a) culturing pine somatic cells in an induction medium to yield embryogenic cells;
(b) culturing the embryogenic cells prepared in step (a) in a maintenance medium to multiply the embryogenic cells and form pre-cotyledonary pine somatic embryos;
(c) culturing pre-cotyledonary pine somatic embryos formed in step (b) in a development medium comprising abscisic acid and an adsorbent composition; and
(d) culturing the pre-cotyledonary pine somatic embryos treated in accordance with step (c) in a development medium that does not include abscisic acid to yield cotyledonary pine somatic embryos.
